(19) United States
(12) Patent Application Publication
(10) Pub. No.: US 2026/0069739 A1
(43) Pub. Date: Mar. 12, 2026

(54) Heat Transfer Apparatus with Ultraviolet Air Treatment

(71) Applicant: Baltimore Aircoil Company, Inc., Jessup, MD (US)

(72) Inventors: Ellie M. Litwack, Columbia, MD (US); David Andrew Aaron, Reisterstown, MD (US); John Arthur Colantonio, Wheat Ridge, CO (US)

(21) Appl. No.: 19/320,082

(22) Filed: Sep. 5, 2025

Related U.S. Application Data
(60) Provisional application No. 63/691,447, filed on Sep. 6, 2024.
(60) Provisional application No. 63/691,451, filed on Sep. 6, 2024.

(51) Int. Cl. A61L 9/20; F28F 25/10; F28G 1/00

(52) U.S. Cl. CPC A61L 9/20; F28F 25/10; F28G 1/00; A61L 2209/111; A61L 2209/12; A61L 2209/16

(57) ABSTRACT

In one aspect, a heat transfer apparatus including an air inlet, an air outlet, a fan assembly to generate an airflow from the air inlet to the air outlet, a heat exchanger to transfer heat to the airflow, and an ultraviolet (UV) light source operable to emit UV light and treat the airflow. The heat transfer apparatus further including an automated cleaning apparatus operable to clean an interior surface of the heat transfer apparatus and increase an intensity of UV light in the heat transfer apparatus. The automated cleaning apparatus has a controller configured to cause the automated cleaning apparatus to clean the interior surface of the heat transfer apparatus in response to a reduced airflow UV treatment effectiveness condition.

### CROSS-REFERENCE TO RELATED APPLICATIONS

This application claims the benefit of U.S. Provisional Application No. 63/691,447, filed Sep. 6, 2024, and U.S. Provisional Application No. 63/691,451, filed Sep. 6, 2024, which are hereby incorporated herein by reference in their entireties.

### TECHNICAL FIELD

This disclosure relates generally to heat rejection apparatuses and, more specifically, to control systems for heat rejection apparatuses.

### BACKGROUND

Heat transfer apparatuses, such as cooling towers, may utilize evaporative cooling to cool airflow and/or a process fluid. The cooling tower may be, for example, an open circuit cooling tower or a closed-circuit cooling tower. The open circuit cooling tower may distribute process fluid such as water onto fill and a fan of the cooling tower generates airflow that directly contacts the process fluid traveling along the fill. The process fluid is cooled via evaporation of some of the process fluid and by interacting with the lower-temperature airflow.

A closed-circuit cooling tower may distribute an evaporative liquid such as water onto an indirect heat exchanger (e.g., a coil, tube-fin, or plate-style heat exchanger) containing a process fluid. The closed-circuit cooling tower has a fan to generate airflow that contacts the indirect heat exchanger. The process fluid inside the indirect heat exchanger is cooled at least in part by a portion of the evaporative liquid absorbing heat from the indirect heat exchanger and with combined sensible and evaporative heat transfer from the process fluid to the indirect heat exchanger then from the indirect heat exchanger to the evaporative fluid. Some indirect heat exchangers may operate in a wet, hybrid or dry mode.

To conserve water, the liquid is often recirculated in cooling towers. The air movement caused by the fan of the cooling tower may carry dirt, debris and airborne liquid particles, also referred to as drift, in the cooling tower and may cause the drift to be emitted from the cooling tower.

The chemical and microbial concentration of the recirculated evaporative liquid may change during operation of the cooling tower. For example, the chemical concentration can increase as water evaporates and the population of microorganisms in the liquid can increase over time. Cooling towers may have liquid treatment systems that add one or more chemicals to a liquid distributed in the cooling tower, such as process fluid or evaporative liquid, to inhibit the growth of biological agents or microbes, such as bacteria, algae, viruses, and/or fungi. An ultraviolet (UV) light source, such as low pressure mercury lamps, may also be used to kill microbes in the air and drift within the cooling tower; however, UV light causes the material of fill sheets, such as a plastic (e.g., PVC) to degrade. To address this issue, some have used light shields in cooling towers to block the UV light from reaching plastic components in the cooling tower. One issue with this approach is that the UV shields may restrict airflow through the cooling tower.

Another shortcoming of UV lights is that low pressure mercury lamps can typically only run for about 10,000 hours before needing to be replaced, which is significantly shorter than the lifespan of the associated cooling tower. As a result, providing a UV light source in a cooling tower may require additional maintenance over the lifespan of the cooling tower.

Moreover, a UV air purification system, when clean, is designed to have adequate UV disinfection performance because surfaces of the heat rejection apparatus, such as interior surfaces of walls of the cooling tower, may reflect the UV light and increase overall UV irradiance within the heat transfer apparatus. However, during operation of the cooling tower, the drift within the cooling tower may deposit water and dissolved solids of the drift on the interior surfaces of the cooling tower. As air travels over the interior surfaces of the cooling tower, the deposited water evaporates, leaving the solids on the interior surfaces of the cooling tower. Additional solids that may accumulate on the interior surfaces of the cooling tower include dirt and dust. The solids on the interior surfaces may include mineral scale such as calcium carbonate. Mineral scale absorbs UV light, which decreases the irradiance of the UV light within the cooling tower and reduces the efficacy of the UV air purification system. The build-up of solids, such as mineral scale and dirt, on the UV lamps may also reduce the efficacy and intensity of the UV light emitted by the UV lamps of the UV air purification system.

### BRIEF DESCRIPTION OF DRAWINGS

FIG. 1 is a schematic diagram of a cooling tower having an ultraviolet (UV) light source;

FIG. 2 is a flow diagram of a method for controlling the UV light source of FIG. 1 based on an input;

FIG. 3 is a flow diagram of a method for controlling the UV light source of FIG. 1 based on two inputs;

FIG. 4 is a flow diagram of a method for controlling the UV light source of FIG. 1 based on an estimated microbial effluence rate of the cooling tower;

FIG. 5 is a flow diagram of a method for controlling the UV light source of FIG. 1 to achieve a desired microbial kill rate;

FIG. 6 is a flow diagram of a method for controlling the UV light source of FIG. 1 to achieve a desired microbial effluence rate of the cooling tower;

FIG. 7 is a flow diagram of a method for detecting a maintenance condition of the UV light source of FIG. 1;

FIG. 8 is a flow diagram of a method for estimating the remaining life of lights of the UV light source of FIG. 1;

FIG. 9 is a flow diagram of a method for detecting when a UV light of the UV light source of FIG. 1 needs maintenance;

FIG. 10 is a flow diagram of a method for operating the cooling tower of FIG. 1 to limit a biological effluence rate of the cooling tower;

FIG. 11 is a flow diagram of a method for operating the cooling tower of FIG. 1 to achieve a minimum microbial kill rate using the UV light source;

FIG. 12 is a flow diagram of a method for initiating a cleaning procedure for the cooling tower of FIG. 1 based on the UV light intensity in the cooling tower;

FIG. 13 is a schematic view of a cooling tower having UV light sources in a plenum and a fan cowl extension of the cooling tower;

FIG. 14 is a cross-sectional view of a member of the cooling tower of FIG. 13 showing a reflective layer secured via adhesive to a surface of the member;

FIG. 15 is a cross-sectional view of a member of the cooling tower of FIG. 13 showing a nonstick layer on a surface of the member;

FIG. 16 is a schematic view of a fan cowl assembly that may be utilized with the cooling tower of FIG. 13;

FIG. 17 is a schematic view of a cleaning apparatus for removing solids from an interior surface and/or a UV light source of a cooling tower;

FIG. 18 is a schematic view of a cleaning apparatus for removing solids from an interior surface and/or a UV light source of a cooling tower;

FIG. 19 is a cutaway view of a portion of a residential HVAC system having a UV air purification system positioned near an evaporator coil of the residential HVAC system;

FIG. 20 is a schematic view of a cleaning apparatus for a residential HVAC system.

### DETAILED DESCRIPTION

In one aspect of the present disclosure, a heat rejection apparatus is provided that comprises a heat exchanger, a liquid distribution system, an airflow generator, a UV light source, and a controller. The liquid distribution system is operable to distribute liquid to the heat exchanger. The airflow generator is operable to move air relative to the heat exchanger. The UV light source is operable to emit UV light (e.g., UV-C light) to kill microorganisms carried in the air and drift within the heat exchanger. The UV light source may include one or more UV lights.

The controller is configured to detect a variable of the heat rejection apparatus and operate the UV light source in a lower energy state or a higher energy state based at least in part on the variable of the heat rejection apparatus. The variable of the heat rejection apparatus may be one or more variables indicative of a microbial effluence condition of the heat rejection apparatus. The variable of the heat rejection apparatus may include, as examples, a fan speed, a drift rate, an airflow rate, an operating status of the liquid distribution system, a biological activity level of the airflow, drift, and/or liquid of the liquid distribution system, and/or an environmental condition such as wind direction, building occupancy, etc. As another example, the variable of the heat rejection apparatus may include a temporal condition such as, for example, a date, season, time of day (e.g., peak cooling hours), day of the week, an operating schedule (e.g., 9 AM-5 PM on business days), and a timer.

Operating the UV light source in the lower energy state includes, for example, turning off the UV light(s), turning off a subset of UV lights, and/or operating the UV light(s) at a lower power level (e.g., operating the UV lights at a lower duty cycle or dimming the UV lights). Operating the UV light source in the higher energy state includes, for example, turning on the UV light(s), turning on a subset of UV lights, and/or operating the UV light(s) at higher power level (e.g., operating the UV lights at a higher duty cycle or brightening the UV lights). Operating the UV light source in the lower energy state prolongs the life of the UV light source compared to conventional approaches of operating UV lights continuously. Operating the UV light source in the lower energy state also reduces the electrical power consumed by the UV light source compared to operation in the higher energy state. Additionally, by operating the UV light source in the lower energy state, the effect of UV light on UV-susceptible material of the heat rejection apparatus may be reduced.

In another aspect of the present disclosure, a heat rejection apparatus is provided that includes a UV light source to eliminate microbes carried in the air and/or drift discharged from the heat rejection apparatus. The heat rejection apparatus includes a controller that controls the UV lights of the UV light source. The controller may operate the UV light source when an air purification condition is present, which may be determined based on data from one or more sensors or inputs. For example, the air purification condition may be present based on measured biological activity (e.g., in the drift, air, or a liquid of the heat rejection apparatus), a drift rate of the heat rejection apparatus, a fan speed of the heat rejection apparatus, an airflow rate of the heat rejection apparatus, environmental conditions (e.g., wind direction), and/or temporal conditions. The controller may adjust operation of UV light source (e.g., turn a subset of the UV lights off and/or dim the UV lights) to provide a desired kill rate (e.g., 90%, 95%, 99%, or 99.99%) of the microorganisms carried in the air leaving the heat rejection apparatus. The controller may turn the UV light source off in absence of the air purification condition. Turning the UV lights off and/or dimming the UV lights in the absence of the air purification condition prolongs the usable life of the UV lights and consumes less electrical power. Further, turning the UV lights off and/or dimming the lights reduces the duration and/or intensity of UV light contacting UV-susceptible material (e.g., PVC fill) which reduces the effect of UV light on the UV-susceptible material. For instance, the UV lights may generally be off or in a lower-power condition and turned on or fully energized periodically (e.g., when the air purification condition is present) to limit the microbial effluence of the heat rejection apparatus. By turning the UV lights off and/or dimming the lights, the effect of UV light on the UV-susceptible material may be reduced.

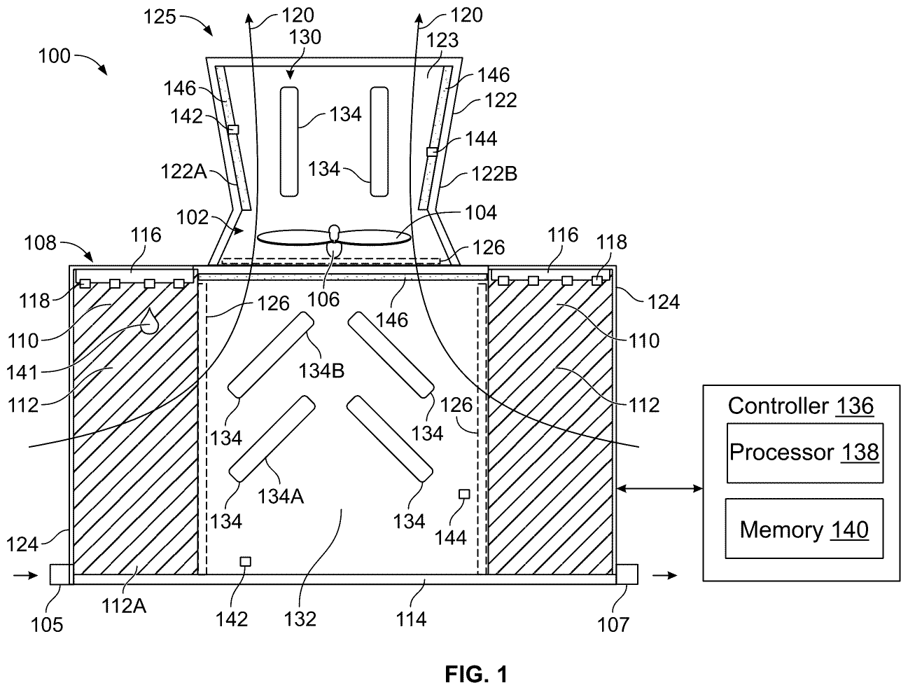

With respect to FIG. 1, a heat rejection apparatus, such as a cooling tower 100, is provided that may be part of a HVAC system of a building. The cooling tower 100 has a process fluid inlet 105 to receive process fluid, such as water, from a heat generating apparatus such as a chiller of a building, a computer datacenter, or another industrial process. The cooling tower 100 removes heat from the process fluid and returns cooled process fluid via a process fluid outlet 107. The cooling tower 100 is an open cooling tower in FIG. 1, but may be a closed circuit cooling tower in another embodiment. The closed circuit cooling tower may be operable in various modes such as dry, wet, hybrid, and/or adiabatic modes.

The cooling tower 100 includes an airflow generator such as a fan assembly 102 including a fan 104 and a motor 106. The cooling tower 100 further includes a liquid distribution system 108 for distributing the process fluid onto a heat exchanger 110. The heat exchanger 110 may be a direct heat exchanger, such as fill 112. In another embodiment, the heat exchanger 110 is an indirect heat exchanger such as a coil that receives process fluid and the liquid distribution system 108 distributes evaporative liquid (e.g., water) onto the coil such that the process fluid is in indirect contact with the evaporative liquid and air flowing about the coil.

The fan 104 generates airflow relative to the fill 112 to cool the process fluid flowing on the fill 112. The cooled process fluid 141 is collected in a receptacle, such as tray or sump 114, of the cooling tower 100. The cooled process fluid may be pumped back to the heat generating apparatus via the outlet 107.

The liquid distribution system 108 includes a header 116 with outlets, such as nozzles 118, through which the process fluid is discharged onto the heat exchanger 110 to cool the process fluid. The header 116 and nozzles 118 operate to evenly distribute the process fluid onto the heat exchanger 110. As the process fluid is distributed onto the heat exchanger 110, the fan assembly 102 operates to cause airflow in the cooling tower 100 relative to the heat exchanger 110. Operation of the fan assembly 102 moves air through the cooling tower along airflow paths 120. The fan assembly 102 draws air in through inlets 124, across the heat exchanger 110, and outward from the cooling tower 100 via an outlet 125. In the embodiment of FIG. 1, the air generally flows in a crossflow direction to the falling process fluid sprayed from the nozzles 118. The cooling tower 100 optionally includes one or more drift eliminators 126 in the section having the fill 112 and/or upstream of the fan 104. The drift eliminator(s) 126 inhibit drift from leaving the cooling tower 100. The cooling tower 100 utilizes a crossflow of air and process fluid relative to the fill 112, although the cooling tower 100 may be provided in different configurations, such as counter flow or parallel flow. In one embodiment, the motor 106 is a variable speed motor capable of rotating the fan 104 at varying speeds. In one embodiment, the motor 106 is a one or two speed motor.

The outlet 125 of the cooling tower 100 includes an upper portion, such as a fan cowl extension 122, that extends downstream of the fan assembly 102. The fan cowl extension 122 directs the flow of air out of the cooling tower 100. In some forms, the fan cowl extension 122 is a velocity recovery device which increases airflow through the cooling tower 100. The fan cowl extension 122 may extend downstream of the fan assembly 102 a length in the range of about 1 foot to about 10 feet. As one example, the fan cowl extension 122 has a length that corresponds to the length of a UV bulb used in the fan cowl extension 122, such as 5½ feet to accommodate one or more UV bulbs having a length of 5 feet. The fan cowl extension 122 may be formed of a metal that reflects ultraviolet (UV) light, such as galvanized steel or aluminum, which may aid in killing microorganisms in the drift of the cooling tower 100 as discussed below. In one embodiment, an interior surface 122A of a side wall 122B of the fan cowl extension 122 has a material attached thereto that reflects UV light, for example, an aluminum tape. In another embodiment, a non-stick coating is applied to the interior surface 122A of the fan cowl extension 122 to inhibit scale and mineral deposits from sticking to the fan cowl extension 122. The non-stick coating may be a material transparent to UV light such as polytetrafluoroethylene (PTFE) to permit the metal of the fan cowl extension 122 to reflect the UV light through the non-stick coating. The non-stick coating allows the interior surface 122A of the fan cowl extension 122 to remain clean, which keeps the reflectivity of the side wall 122B of the fan cowl extension 122 at a high value.

The cooling tower 100 includes a UV light source 130 downstream of the heat exchanger 110 and downstream of fan 104. For example, the UV light source 130 may include one or more UV lights 134 of a plenum 132 of the cooling tower 100 and/or one or more UV lights 134 of the fan cowl extension 122. The fan cowl extension 122 keeps the air moved by the fan assembly 102 near the UV lights 134 rather than blowing away after exiting the fan assembly 102. The fan cowl extension 122 reflects the UV light back into the airflow to increase the irradiance of the UV lights 134. The fan cowl extension 122 also directs the UV light 134 upward and away from UV-susceptible material that may be outward of the cooling tower 100. In one embodiment, the cooling tower 100 does not include a fan cowl extension 122 and the UV lights 134 thereof.

The plenum 132 may be formed of materials similar to the fan cowl extension 122 discussed above, for example, one or more materials that reflect UV light (e.g., aluminum, galvanized steel) and/or provide a non-stick coating (e.g., PTFE). The UV lights 134 emit UV light when turned on. In one embodiment, the UV lights 134 emit light including light in the UV-C spectrum (e.g., having a wavelength in the range of about 200 nm to about 280 nm) to kill microorganisms in the drift of the cooling tower 100.

The UV lights 134 may include, as examples, low pressure mercury lamps, medium pressure mercury lamps, excimer lamps, and/or UV LEDs. Low pressure mercury lamps operate with a mercury pressure in the range of about 100 Pascals (Pa) to about 1000 Pa. Low pressure mercury lamps emit UV-C light that kills microorganisms. The UV lights 134 may be turned on/off and, in one embodiment, are dimmable, for example, by adjusting the duty cycle of the UV lights 134.

UV-C light is effective at killing microorganisms when the microorganisms are exposed to a threshold average radiant exposure of UV-C light. Radiant exposure is irradiance of the UV light (the flux of radiant energy per unit area) multiplied by an air residence time such as the time the air is exposed to the UV light. For example, for coliform bacteria, a 90% kill rate may be achieved by operating the UV lights 134 to output an average radiant exposure of about 0.625 millijoules (mJ)/cm² to the air flowing through the cooling tower 100. To achieve a 99% kill rate for coliform bacteria, the UV lights 134 may be operated to output an average radiant exposure of about 1.25 mJ/cm² to the air. To achieve a 99.99% kill rate for coliform bacteria, the UV lights 134 may be operated to output an average radiant exposure of about 2.5 mJ/cm² to the air. The UV lights 134 may be within the airflow path 120 of the cooling tower 100 to expose the air flowing in the cooling tower 100 to UV light at the desired intensity levels. For example, the UV lights 134 may be spaced apart from one another in the airflow path 120 to radiate UV light at different locations in the airflow path 120. Using multiple UV lights 134 along the airflow path 120 permits one or more of the UV lights 134 to be turned off when a subset of the UV lights 134 provide an adequate kill rate. Turning off the UV lights 134 (e.g., some or all) reduces the power consumed by the UV light source 130 and prolongs the lifespan of the UV lights 134 being turned off. For instance, UV lights 134 have a limited lifespan, for example, a limited number of hours (e.g., 10,000 hours for a low pressure mercury lamp) that the UV light 134 can operate for before failing (e.g., burning out). By turning a UV light 134 off, the UV light 134 can be operated periodically and over a longer period of time. By turning some or all of the UV lights 134 of the UV light source 130 off, the UV light source 130 can thus be operated over a longer period of time, for example, over the lifespan of the cooling tower 100. Moreover, by turning off the UV lights 134, the plastic degradation caused by the UV light source 130 is reduced. For example, by turning off UV lights 134A, 134B near the fill sheets 112A, the fill sheets 112A are subjected to less UV light than if the UV lights 134A, 134B were turned on.

Some portions of the cooling tower 100 may also reflect UV light output from the UV lights 134, which aids in killing the microorganisms in the drift of the cooling tower 100. For example, the cooling tower 100 includes the UV lights 134 in the fan cowl extension 122. The inner surface 122A of the fan cowl extension 122 reflects the UV light emitted from the UV light lights 134 of the fan cowl extension 122 back into the airflow path 120. The reflectivity of the interior surface 122A of the fan cowl extension 122 increases the number of times a UV light wave travels across a central opening 123 of the fan cowl extension 122 and interacts with the drift carried in the air traveling through the central opening 123, which increases the efficacy of the UV light purification provided by the cooling tower 100. As noted above, the interior surface 122A of fan cowl extension 122 can be formed of a metallic material that reflects UV light. For example, aluminum has a UV light reflectivity in the range of about 70% to about 90%. Galvanized steel has a UV light reflectivity of about 50%. The walls and other structural members of the cooling tower 100 that have surfaces defining the plenum 132 of the cooling tower 100 may similarly reflect UV light from UV lights 134 in the plenum 132 to improve the efficiency of air purification in the plenum 132. Over time, scale and mineral deposits 146 from the drift may build up on the surfaces of the cooling tower 100 defining the airflow path 120. The scale and mineral deposits reflect less than 40% of UV light and thus reduce the amount of UV light reflected by the cooling tower 100.

The UV light source 130 may be operated taking into account the effect of the reflected UV light has on the microbial kill rate. For example, the power emitted by the UV lights 134 of the UV light source 130 may be increased or decreased based upon the reflectivity of surfaces within the cooling tower 100 to achieve the desired microbial kill rate. For example, as mineral deposits 146 increase, the UV light source 130 may be operated in a higher power mode, e.g., by turning on additional UV lamps, to compensate for the reduced reflectivity of the internal surfaces.

The UV light source 130 includes or is in communication with a controller 136 to control operation of the UV light source 130. The controller 136 includes a processor 138 in communication with memory 140. The processor 138 may execute programs and functions stored in the memory 140 to control operations of the UV light source 130. The processor 138 may also execute programs and functions stored in the memory 140 to control other operations of the cooling tower 100 or another system, such as an HVAC system. The processor 138 may include, as examples, a microprocessor, an application-specific integrated circuit (ASIC), or a field programmable gate array (FPGA). The memory 140 may include, as examples, an electrical charge-based storage media such as EEPROM or RAM, ROM, or other non-transitory computer readable media such as a flash memory device or magnetic or optical storage medium. In some embodiments, the controller 136 is part of the UV light source 130 and is configured to communicate with another controller of the cooling tower 100 and/or a master controller of the HVAC system. In some embodiments, the controller 136 is a controller of the cooling tower 100 configured to control the UV lights 134 of the UV light source 130 and the fan assembly 102. In some embodiments, the controller 136 is the master controller of the building HVAC system configured to communicate with one or more cooling towers 100 to control operation of the UV light sources 130 of the cooling towers 100. For example, the controller 136 may control the operation the UV light source 130 in multiple cooling towers of a building to achieve a desired microorganism kill rate or biological effluence rate across the cooling towers (e.g., turning the UV light sources 130 on in some of the cooling towers and off in others).

The controller 136 may be in communication with one or more sensors and operate the UV light source 130 of the cooling tower 100 based on data from the one or more sensors. The cooling tower 100 may include one or more biological activity sensors 142 that may be used to detect a biological activity level in a liquid sprayed and collected in the cooling tower, a biological activity level in the drift, and/or a biological activity level in the airflow.

In one embodiment, the sump 114 includes a biological activity sensor 142 to measure the biological activity levels of the liquid in the sump 114. Additionally or alternatively, the plenum 132 and/or fan cowl extension 122 may include a biological activity sensor 142 to measure the biological activity levels in the air and/or drift in the air. The biological activity level may be a measurement of a quantity of colony forming units (CFUs) per volume of air or water in the sample tested. The biological activity sensors 142 may measure the biological activity levels according to various approaches. As one example, the biological activity sensor 142 detects a bacteria count (e.g., quantity of any type of bacteria or quantity of a particular type of bacteria) using a plate count method. As one example, the biological activity sensor 142 detects an adenosine triphosphate (ATP) level, for example, using an AquaSnap® test. As one example, the biological activity sensor 142 detects the levels of DNA from a particular microbe in a sample, for example, using polymerase chain reaction (PCR) testing. As one example, the biological activity sensor 142 is a biofilm sensor that detects biofilm levels. As one example, the biological activity sensor 142 uses spectrophotometric (turbidimetric) analysis to determine the quantity of bacteria in a sample. As one example, the biological activity sensor 142 uses radiometric analysis to determine the quantity of bacteria in a sample. As one example, the biological activity sensor 142 uses an impedance measurement to determine the quantity of bacteria in a sample. As one example, the biological activity sensor 142 uses a Direct Fluorescent Antibody method or other antibody tests to determine the quantity of bacteria in a sample. As one example, the biological activity sensor 142 includes graphene-based nano-sensors to detect biological activity levels.

The cooling tower 100 may include a UV light sensor 144 that detects an intensity of the UV light emitted by the UV light source 130, such as by detecting a radiant exposure of the UV light. The cooling tower 100 may include multiple UV light sensors 144 to measure the intensity of the UV light at various locations of the cooling tower 100, for example, at multiple positions about the fan cowl extension 122 and/or in the plenum 132. The controller 136 may use the UV light sensor 144 to determine the intensity of the UV light in the cooling tower 100. For instance, the intensity of the UV light may decrease as scale and mineral deposits 146 build up on the interior surfaces of the cooling tower 100 and decrease the reflectivity of the interior surfaces. The controller 136 may use the UV light sensor 144 to determine whether the UV lights 134 are operational. For example, the controller 136 may energize one UV light 134 and determine if that one UV light 134 is operational based on whether a threshold level of UV light is detected. The controller 136 may similarly cycle through all of the UV lights to determine which UV lights 134 are operational and which need to be replaced (e.g., because they have burned out). The controller 136 may account for UV lights 134 determined to not be operational when operating the UV light source 130, for example, to output a desired UV light intensity. As an example, the controller 136 may turn on one or more additional UV lights 134 to compensate for each UV light that is non-operational. Upon detecting that one or more UV lights 134 are not operational, the controller 136 may output an alert of a maintenance condition in the UV light source 130. The maintenance condition may indicate which UV light(s) 134 have burned out or need to be replaced. The maintenance alert may be, for example, a siren, a light, a SMS text, an email, and/or a signal to a remove computer that initiates an application notification in a maintenance worker's smartphone or other portable electronic device, as some examples. In one embodiment, the UV light source 130 includes a redundant UV light 134 that the controller 136 may operate upon determining a UV light 134 is no longer operational.

The controller 136 may also receive data, such as variables, of operating conditions of the cooling tower 100 including a fan speed of the fan assembly 102, a drift rate of the cooling tower 100, and/or an airflow rate through the cooling tower 100. The fan speed may be a variable selected based on the load of the cooling tower 100. For example, a controller of the cooling tower 100 or master controller of an HVAC system sets the speed at which the fan assembly 102 operates. The drift rate may be detected by a drift sensor that measures the amount of drift in the airflow generated by the fan assembly 102. The drift rate may be calculated based on the operating conditions of the cooling tower 100, for example, the fan speed, airflow rate, the chemistry of the liquid distributed in the cooling tower 100, etc. The airflow rate may be detected by a sensor that measures the speed of the air. Alternatively or additionally, the airflow rate may be calculated based on the fan speed of the fan assembly 102. In one embodiment, at some lower fans speeds, drift is known to not leave the cooling tower 100 and the UV lights 134 can remain completely off during these known lower fans speeds as determined by controller 136.

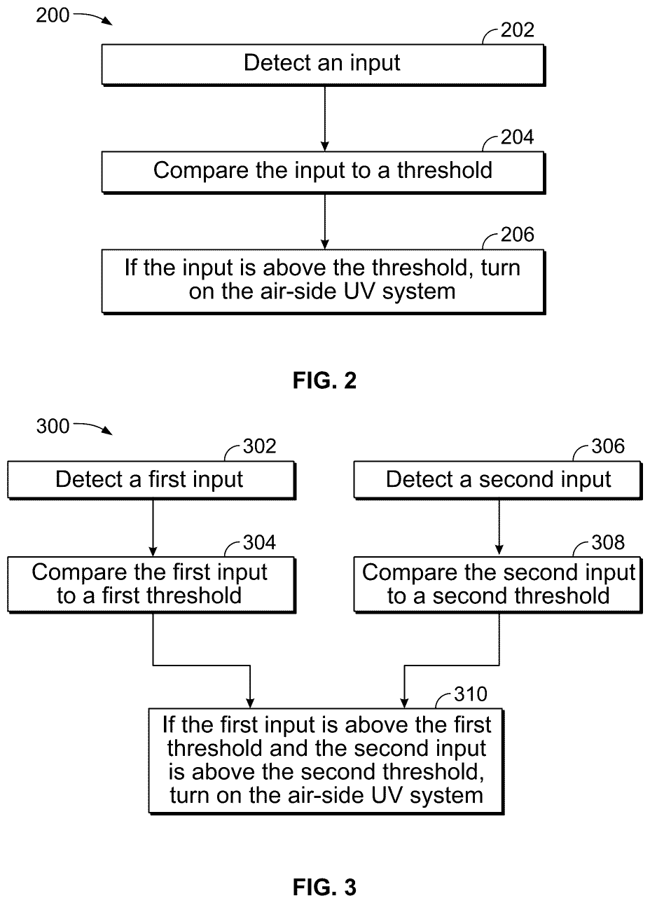

With respect to FIG. 2, the controller 136 may operate the UV light source 130 according to method 200 when an air purification condition is present. In method 200, the controller 136 detects 202 an input indicative of the air purification condition. The input may be an operating variable of the cooling tower 100 or based on data received from one or more of the sensors 142. The input may be, as examples, a fan speed of the fan assembly 102, a drift rate of the cooling tower 100, an airflow rate of the cooling tower 100, operation of a pump of the cooling tower 100, a biological activity level in the heat rejection liquid, a biological activity level in the drift, a biological activity level in the airflow, an environmental condition such as wind direction, building occupancy, etc., or a temporal condition such as, for example, a date, season, time of day (e.g., peak cooling hours), day of the week, an operating schedule (e.g., 9 AM-5 PM on business days), and a timer (e.g., time since the cooling tower 100 was last maintained).

The controller 136 compares 204 the input to a threshold. The threshold may be a value stored in memory 140 that is selected to trigger operation of the UV light source 130. The threshold may be a value selected to limit effluence of microorganisms from the cooling tower 100 when certain conditions are present. For example, the conditions may indicate that microorganism effluence is likely to be higher, for example, due to increased biological activity and/or the increased volume of air emitted from the cooling tower 100. For example, the threshold may be a threshold fan speed, threshold drift rate, threshold airflow rate, threshold heat rejection liquid biological activity level, threshold drift biological activity level, or threshold airflow biological activity level. As another example, the threshold may be a threshold environmental condition. For example, that the wind direction is toward a stream or populated area or that building or surrounding buildings are presently occupied. The step 204 may involve determining whether the input is greater than, equal to, or less than a threshold as one example.

If the controller 136 determines that the input meets or exceeds the threshold, the controller 136 turns on the UV light source 130 at step 206 to kill the microorganisms carried in the drift of the cooling tower 100. Where the input does not meet or exceed the threshold, the controller 136 sets the UV light source 130 to be off. As an example, the input may be a fan speed and the threshold fan speed may be a fan speed below which it is known that drift is not being discharged from the cooling tower 100. In this approach, the cooling tower 100 operates the UV light source 130 when a certain air purification condition is present, for example, where microorganism effluence may be high or above a threshold. By not operating the UV lights 134 continually, the life of the UV lights 134 may be prolonged and degradation of UV-sensitive material in the cooling tower 100 is reduced. In some embodiments, the controller 136 determines a UV light intensity for the UV light source 130 when the UV light source is turned on. The UV light intensity may be determined based on the input and/or additional inputs. The fan speed may be determined via fan control system feedback or detecting the fan speed from a variable frequency drive of the fan assembly 102.

The controller 136 may turn on the UV light source 130 in real-time or at a later time upon determining that the input meets or exceeds the threshold. For example, the controller 136 may determine that the biological activity level of the heat rejection liquid exceeds a threshold when the cooling tower 100 is off or operating with a lower fan speed and turn on the UV light source 130 later when the cooling tower 100 is operating with a higher fan speed. The controller 136 may use the input data to predict future conditions of the cooling tower 100 and determine when to operate the UV light source 130 based on the predictions. For example, the controller 136 predicts a future biological activity level in the heat rejection liquid and turns on the UV light source 130 at a time when biological activity level in the heat rejection liquid is predicted to exceed a threshold. The controller 136 may detect trends (e.g., an increase in biological activity level in the heat rejection liquid) in the input data and determine when to operate the UV light source 130 based on the trends. The controller 136 may use historical data of the cooling tower 100 to predict future conditions of the cooling tower 100 and/or determine when to operate the UV light source 130. For example, the controller 136 may predict that the cooling tower 100 will be operating with a fan speed above a threshold rate during a period of time (e.g., 12 PM to 5 PM) and determine to operate the UV light source 130 during that time period based on the current and/or future predicted biological activity level in the heat rejection liquid. The controller 136 may receive weather data (e.g., ambient temperature data, seasonal data) and determine when to operate the UV light source 130 based on predicted operation of the cooling tower 100 given the weather conditions.

With respect to FIG. 3, the controller 136 may operate the UV light source 130 according to method 300 to turn on the UV light source 130 when an air purification condition is present, where the air purification condition is comprised of multiple sub-conditions. In method 300, the controller 136 detects 302 a first input and compares 304 the first input to a first threshold similar to step 204 of method 200. The controller 136 also detects 306 a second input different from the first input and compares 308 the second input to a second threshold similar to step 204 of method 200. The first and second inputs could be any of the inputs provided above with respect to method 200. As an example, the first input is a drift rate and the second input is a variable indicative of the biological activity in the drift. As another example, the first input is a variable indicative of the biological activity in the drift and the second input is an airflow rate in the cooling tower 100. If the first input is above the first threshold and the second input is above the second threshold, the controller 136 turns on the UV light source 130 at step 310 to kill the microorganisms carried in the airflow out of the cooling tower 100. Where the first input does not exceed the first threshold and/or the second input does not exceed the second threshold, the controller 136 may set the UV light source 130 to be off. Some sub-conditions may be met when the input is below a threshold or when a certain condition is present (e.g., wind direction). While this method uses two inputs, in other forms, the controller 136 may turn on the UV light source 130 based on three, four, or more inputs.

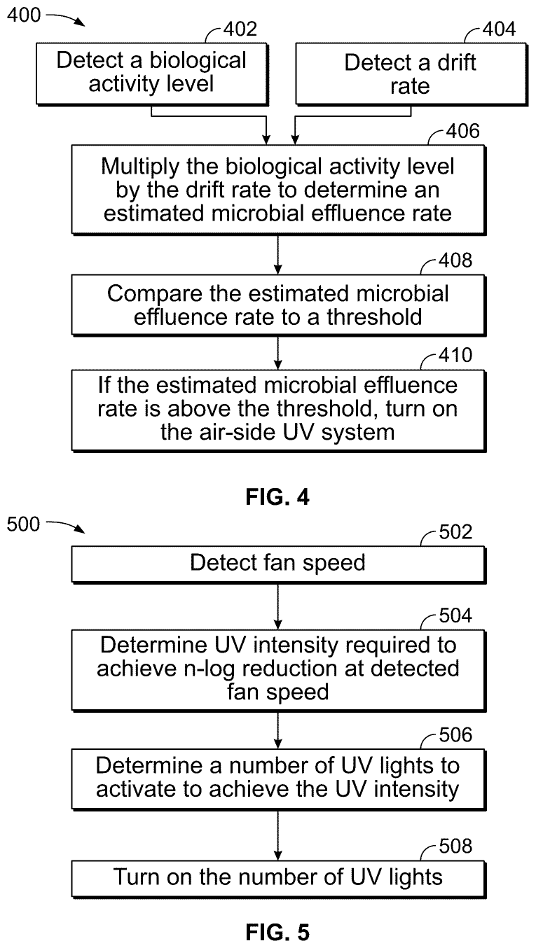

With respect to FIG. 4, the controller 136 may operate the UV light source 130 according to method 400 based on the microbial effluence rate. The controller 136 detects 402 a biological activity level in the cooling tower 100. The controller 136 may detect the biological activity level using data from the biological activity sensor 142. The biological activity level may be the biological activity level in the air, drift, and/or liquid distributed in the cooling tower 100. The controller 136 also detects 404 a drift rate of the cooling tower 100. The controller 136 may detect the drift rate based on a speed of the fan assembly 102 as discussed above.

The controller 136 estimates 406 a microbial effluence rate of the cooling tower 100 based on the detected biological activity level and the detected drift rate. In one approach, the controller 136 estimates the microbial effluence rate by multiplying the biological activity level by the drift rate. The controller 136 compares 408 the estimated microbial effluence rate of the cooling tower 100 to a threshold microbial effluence rate value. The threshold microbial effluence rate value may be stored in the memory 140. The threshold microbial effluence rate value may be set based on applicable regulations or, for example, set by the operator of the cooling tower 100 to limit the microbial effluence rate of the cooling tower 100.

If the estimated microbial effluence rate exceeds the threshold microbial effluence rate value, the controller 136 operates 410 the UV light source 130 to kill microbes carried in the airflow of the cooling tower 100. For example, the controller 136 may turn on all of the UV lights 134 of the UV light source 130. As another example, the controller 136 may operate the UV light source 130 to achieve a desired kill rate (e.g., 90%, 99%, 99.9%, 99.99%), for example, to reduce the microbial effluence rate of the cooling tower 100 to a desired value. The controller 136 may, for example, operate a subset of the UV lights 134 and/or dim some or all of the UV lights 134 to achieve the desired kill rate. Where the estimated microbial effluence rate does not exceed the threshold microbial effluence rate value, the controller 136 may set the UV light source 130 to be off.

With respect to FIG. 5, the controller 136 may operate the UV light source 130 according to method 500 to achieve a desired kill rate of microbes in the air emitted from the cooling tower 100. The controller 136 detects 502 a fan speed of the fan assembly 102. The speed of the fan assembly 102 may be set, for example, upon the load of the cooling tower 100. The controller 136 may detect the fan speed, for example, by receiving the fan speed from another controller of the cooling tower 100, by retrieving the fan speed from memory 140, or detecting the power consumption of the fan assembly 102.

The controller 136 determines 504 a UV light intensity to achieve a desired reduction of microbes in the drift of the cooling tower 100. For example, the cooling tower 100 may be set to achieve a certain n-log kill rate of the microbes. For instance, a 1 log kill is a 90% reduction, a 2 log kill is a 99% reduction, a 3 log kill is a 99.9% reduction, etc. As one example, the controller 136 may reference a formula or lookup table (e.g., stored in memory 140) that correlates UV light intensity and microbe reduction. As discussed above, a certain percentage of microbes are killed upon applying a certain intensity of UV light energy to a volume of air. The volume of air flowing through the cooling tower 100 is proportional to the speed of the fan assembly 102. Therefore, a higher fan speed causes a higher volume of air to flow through the cooling tower 100, which results in a higher UV light intensity being needed to apply the requisite amount of UV light intensity to the air and drift carried therewith before the drift flows out of the cooling tower 100. Conversely, a lower fan speed causes a lower volume of air to flow through the cooling tower 100, which results in a lower UV light intensity being needed to apply the requisite amount of UV light intensity to the air and drift carried therewith because the air is exposed to the UV light for a longer period of time before flowing out of the cooling tower 100.

The controller 136 determines 506 how many UV lights 134 of the UV light source 130 to activate to achieve the determined UV light intensity. The controller 136 may determine how many of the UV lights 134 to turn on based on the rated output for each UV light 134. The controller 136 may reference a formula or lookup table (e.g., stored in memory 140) that correlates a number of UV lights 134 to UV light intensity. The controller 136 may refer to UV light intensity data of the cooling tower 100 collected by the UV light sensor 144 from previous operations that associate the UV intensity with the number of UV lights 134 that are turned on. Using data of the UV light sensor 144 may account for the reflectivity of the interior surfaces of the cooling tower 100 (e.g., the fan cowl extension 122) as well as any scale or mineral deposit buildup reducing the reflectivity in the cooling tower 100. In some forms, the controller 136 may turn on some or all of the UV lights 134 and measure the UV light intensity using the UV light sensor 144. For example, the controller 136 may turn on one UV light 134 and turn on additional UV lights 134 until the desired UV light intensity is achieved. As another example, the controller 136 may turn on the UV lights 134 and adjust the dimming level of the UV lights 134 until the desired UV light intensity is achieved.

Upon determining how many UV lights 134 to turn on at step 506, the controller 136 activates or energizes the determined number of UV lights 134 at step 508 to achieve the determined UV light intensity. The controller 136 may activate a subset of the UV lights 134 so that not all of the UV lights are turned on, preserving the usable life of the unused UV lights 134. The controller 136 may track which UV lights 134 are turned on and how long each UV light 134 is operated during operation of the cooling tower 100. The controller 136 may select which UV lights 134 to turn on based on how long each has been operated, for example, so that all of the UV lights 134 reach the end of their usable life at approximately the same time.

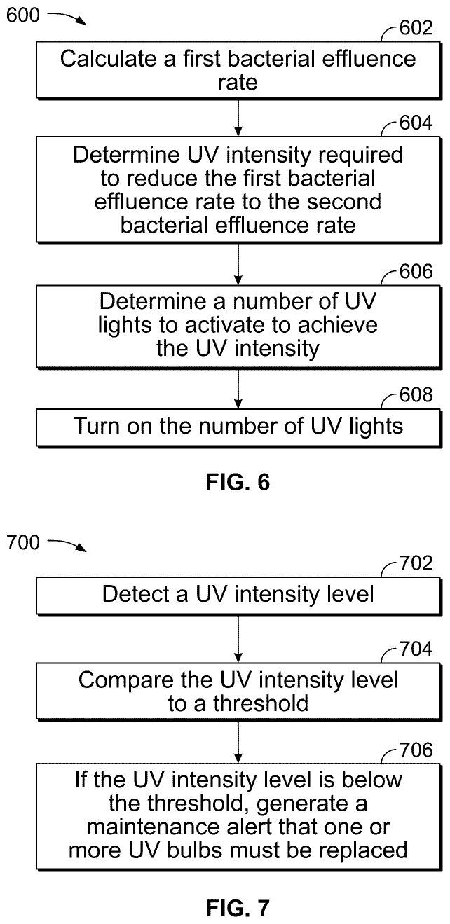

With respect to FIG. 6, the controller 136 may operate the UV light source 130 according to method 600 to achieve a desired kill rate of microorganisms in the drift of the cooling tower 100. The controller 136 calculates 602 a current or first bacterial effluence rate of the cooling tower 100, for example, using the approaches discussed above. The controller 136 determines 604 the UV light intensity needed to reduce the current bacterial effluence rate to a target or second bacterial effluence rate. For example, the controller 136 determines the UV light intensity at step 604 based on the biological activity level in the cooling tower 100, the drift rate, the fan speed, and/or the airflow rate of the cooling tower 100.

The controller 136 determines 606 how many UV lights 134 of the UV light source 130 to activate (e.g., turn on) to achieve the determined UV light intensity. The controller 136 may determine the number of UV lights 134 to turn on to achieve a certain UV light intensity as discussed above (e.g., in step 506). Upon determining 606 how many UV lights 134 to turn on, the controller 136 activates 608 the determined number of UV lights 134. By turning on these UV lights 134, the current bacterial effluence rate is reduced to the target bacterial effluence rate.

With respect to FIG. 7, the controller 136 may detect a maintenance condition of the UV light source 130 using method 700, for example, when a UV light 134 is not able to be turned on. The UV light 134 may not turn on, for example, because the UV light 134 has burned out. The controller 136 detects 702 a UV intensity level in the cooling tower 100. The controller 136 may detect the UV intensity level with the UV light sensor 144 when the UV lights 134 of the UV light source 130 are turned on. The controller 136 compares 704 the detected UV intensity level to a threshold intensity value. The threshold intensity value may be stored in memory 140. The threshold intensity value may be a UV intensity detected by the UV light sensor 144 from when the UV lights 134 are known to be functioning properly, e.g., when the cooling tower 100 was first installed. If the detected UV intensity level is below the threshold intensity value, the controller 136 may output 706 an alert of a maintenance condition in the UV light source 130 indicating that the cooling tower 100 needs maintenance or needs servicing. The maintenance condition may indicate that one or more of the UV lights 134 have burned out or need to be replaced. The maintenance alert may also indicate that the cooling tower 100 needs to be cleaned. For example, scale and mineral deposits 146 on the internal surfaces of the fan cowl extension 122 may reduce the reflectivity of the fan cowl extension 122, bringing the UV intensity level in the cooling tower 100 below the threshold intensity value. The maintenance alert may indicate to clean the scale and mineral deposits from the cooling tower 100. The maintenance alert may be, for example, a siren, a light, a SMS text, an email, and/or a signal that initiates an application notification in a maintenance worker's smartphone, as some examples.

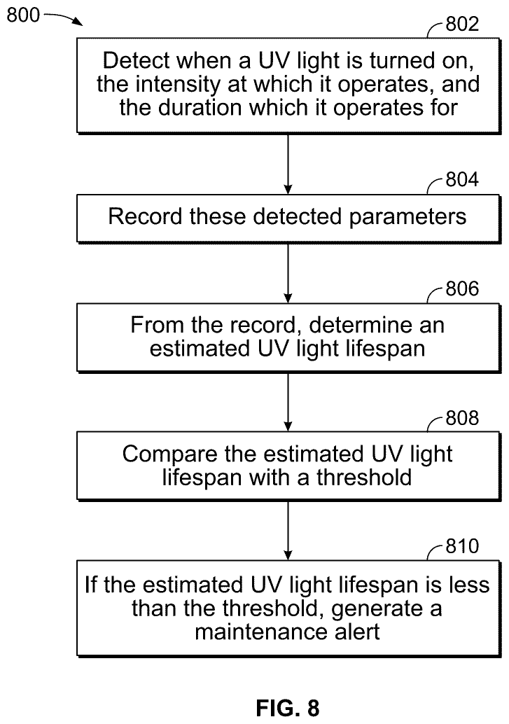

With respect to FIG. 8, the controller 136 may use method 800 to generate a maintenance alert when one or more of the UV lights 134 reach the end of an estimated lifespan of the one or more UV lights 134. In one embodiment, for each UV light 134, the controller 136 detects 802 operating parameters of the UV light 134 such as when the UV light 134 is on and the duration the UV light 134 is on. The controller 136 may also detect the intensity at which the UV light 134 operates when turned on, for example, whether the UV light 134 is dimmed or operated at less than full power. The controller 136 records or stores the detected operating parameters at step 804 to the UV light 134. For example, the operating parameters for each UV light 134 may be stored in memory 140.

The controller 136 estimates 806 the lifespan for one or more of the UV lights 134 based on the recorded operating parameters. For example, the UV lights 134 may be on for a certain period of time (e.g., 10,000 hours) before burning out. Dimming UV lights 134 that are low pressure mercury bulbs when they are on may shorten the lifespan of the bulb.

Upon estimating the lifespan of the one or more UV lights 134, the controller 136 may compare 808 the estimated lifespan for the one or more UV lights 134 to a threshold. The threshold may be, for example, 100 hours. The threshold may be selected to provide a maintenance alert that a UV light 134 is going to fail. The threshold may be a period of time sufficient to enable maintenance personnel to replace the UV light 134 before failure. If the estimated lifespan of the UV light 134 is below the threshold, the controller 136 may output 810 a maintenance alert that the UV light 134 is going to fail soon. The maintenance alert may notify maintenance personnel that the UV light 134 is reaching the end of its estimated lifespan and to replace the UV light 134.

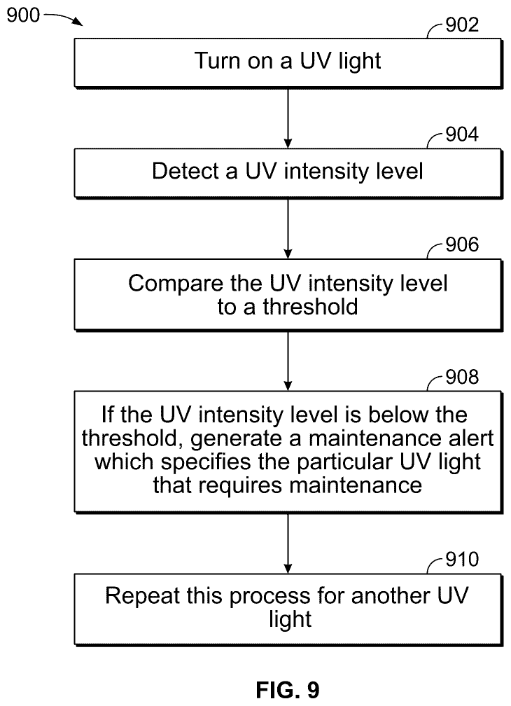

With respect to FIG. 9, the controller 136 may use method 900 to determine whether a particular UV light 134 needs maintenance. In the method 900, the controller 136 activates 902 one UV light 134 of the UV light source 130. The controller 136 detects 904 a UV intensity level in the cooling tower 100, for example, using the UV light sensor 144. The controller 136 compares 906 the UV intensity level to a UV intensity threshold, for example, a UV intensity threshold indicative of whether the UV light 134 is emitting UV light. The UV intensity threshold may be stored in memory 140. As one example, the UV intensity threshold is 80% of the UV intensity measured when the light was new or first installed.

If the UV intensity level is below the threshold, the controller 136 determines that the UV light 134 no longer has adequate UV light output (e.g., the UV light 134 is not on or the intensity has faded substantially) and is in need of maintenance. For example, the controller 136 may determine that the UV light 134 has it has reached the end of its lifespan and has burned out. The controller 136 outputs 908 a maintenance alert that specifies the particular UV light 134 that needs maintenance. The controller 136 may repeat 910 these steps for each UV light 134 of the UV light source 130 to determine which UV lights 134 are operational and which need maintenance. The controller 136 may run method 900 periodically (e.g., weekly) to check the status of each UV light 134. The controller 136 may record which UV lights 134 are not operational and activate the UV lights 134 determined to be operational when subsequently operating the UV light source 130. When the UV lights 134 are replaced, the technician may provide input to the controller 136 indicating which UV lights 134 were replaced.

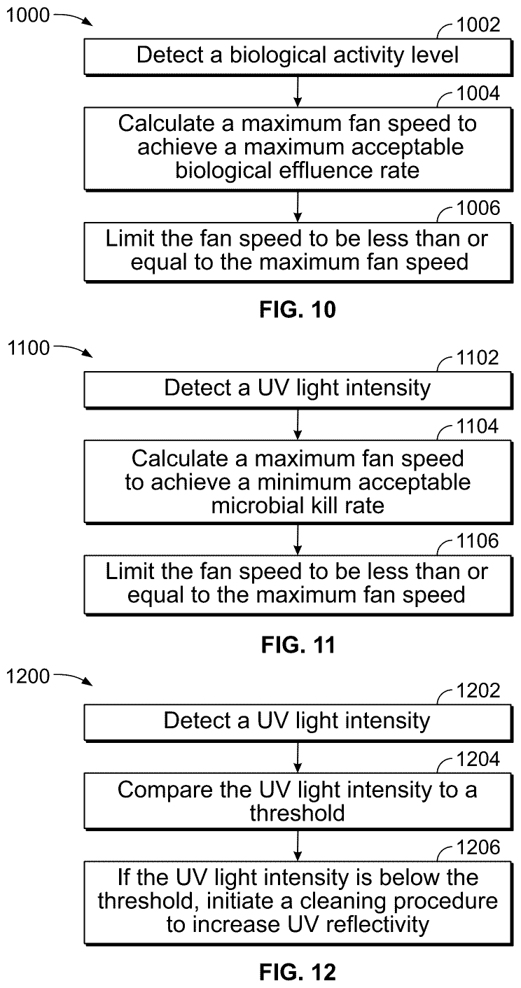

With respect to FIG. 10, the controller 136 may use method 1000 to control the biological effluence rate from the cooling tower 100. The controller 136 detects 1002 a biological activity level of the cooling tower 100 as discussed above. For example, the controller 136 may use a biological activity sensor 142 to measure the biological activity level in the air, drift, and/or liquid distributed and collected in the cooling tower 100. The controller 136 calculates 1004 a maximum fan speed at which the fan assembly 102 can be operated without the biological effluence rate of the cooling tower 100 exceeding a maximum acceptable biological effluence rate. The controller 136 may calculate 1004 the maximum fan speed based on the estimated airflow rate at the fan speed and at the current the biological activity levels. The controller 136 may also account for the kill rate (e.g., 90%) of the microorganisms from the UV light source 130 when estimating the biological effluence rate of the cooling tower 100. The controller 136 limits 1006 the speed of the fan assembly 102 to the calculated maximum fan speed to ensure the biological effluence rate does not exceed the maximum acceptable biological effluence rate.

With respect to FIG. 11, the controller 136 may use method 1100 to control the microbial kill rate of the cooling tower 100. The controller 136 detects 1102 a UV light intensity of the cooling tower 100 as discussed above. For example, the controller 136 may use the UV intensity sensor 144 to measure the intensity of the UV light in the cooling tower 100. The UV intensity sensor 144 may measure the intensity of the UV light emitted from the UV light source 130 and reflected in the cooling tower, for example, in the fan cowl extension 122 and/or plenum 132. The controller 136 calculates 1104 a maximum fan speed at which the fan assembly 102 can be operated to achieve a minimum acceptable microbial kill rate (e.g., 1 log, 2 log, 3 log, etc.). For instance, as discussed above, the longer the air carrying the microorganisms is exposed to the UV light at a certain intensity, the greater the kill rate. The controller 136 may calculate the maximum fan speed based on the estimated airflow rate at the fan speed and the UV light intensity in the cooling tower 100. The controller 136 limits 1106 the speed of the fan assembly 102 to the calculated maximum fan speed to ensure the minimum acceptable microbial kill rate is achieved.

With respect to FIG. 12, the controller 136 may use method 1200 to determine whether the cooling tower 100 needs to be cleaned. The controller 136 detects 1202 a UV light intensity in the cooling tower 100. The controller 136 may detect the UV light intensity using the UV light sensor 144. The controller 136 compares 1204 the UV light intensity to a threshold intensity value. The threshold intensity value may be stored in memory 140. The threshold intensity value may be a value known to indicate that the reflectivity of the internal surfaces of the cooling tower 100, such as one or more interior surfaces of the fan cowl extension 122, has decreased significantly, for example, due to scale and mineral deposit buildup. If the UV light intensity is below the threshold intensity value, the controller 136 initiates 1206 a cleaning procedure to restore the reflectivity of the cooling tower 100. For example, the cleaning procedure may remove the scale and mineral deposits 146 from the fan cowl extension 122 and/or plenum 132. In some forms, the cooling tower 100 includes spray nozzles positioned to spray one or more internal surfaces of the fan cowl extension 122 and/or plenum 132. When the controller 136 initiates the cleaning procedure, clean water may be sprayed from the spray nozzles onto the fan cowl extension 122 and/or plenum 132 to remove the scale and mineral deposits 146 and increase UV reflectivity. In some forms, the controller 136 issues a maintenance alert that the cooling tower 100 needs cleaning to prompt manual cleaning of the cooling tower 100 to restore the reflectivity of the cooling tower 100 (e.g., one or more internal surfaces of the fan cowl extension 122).

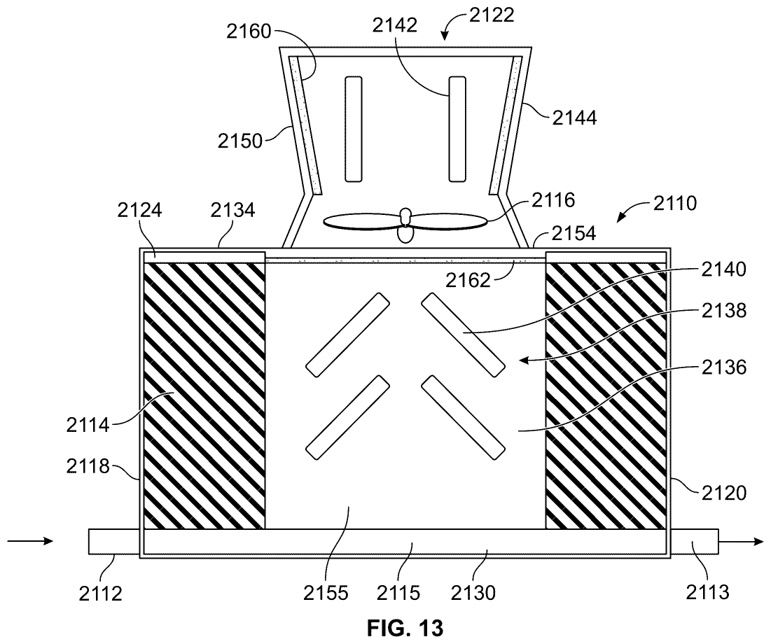

Regarding FIG. 13, a heat transfer apparatus such as a cooling tower 2110 is provided that includes an inlet 2112 for receiving a heated process fluid 2115 and an outlet 2113 for directing cooled process fluid 2115 back to an industrial process, such as an HVAC system, a computer datacenter, or a manufacturing process as some examples. The cooling tower 2110 has a fan 2116 operable to generate air flow from air inlets 2118, 2120 to an air outlet 2122 of the cooling tower 2110. The cooling tower 2110 has a liquid distribution system 2124 operable to distribute the heated process fluid 2115 onto a heat exchanger such as fill sheets 2114. The process fluid 2115 is cooled by air flow across the fill sheets 2114 and is collected in a tray or sump 2130 of the cooling tower 2110.

The cooling tower 2110 has a housing, such as an outer structure 2134, that houses the components of the cooling tower 2110, a plenum 2136 downstream of the fill sheets 2114, and one or more UV light sources 2138 such as UV light sources 2140 in a plenum 2136 and UV light sources 2142 in a fan cowl extension 2144 of the outlet 2122. The one or more UV light sources 2138 may include low pressure mercury lamps, medium pressure mercury lamps, excimer lamps, and/or UV LEDs. Low pressure mercury lamps operate with a mercury pressure in the range of about 100 Pascals (Pa) to about 1000 Pa. UV light sources 2138, such as low pressure mercury lamps, emit UV-C light that kills microorganisms. In one embodiment, one or more of the UV light sources 2140, 2142 are elongated with their lengths oriented parallel to the direction of airflow in the cooling tower 2110 to maximize UV exposure as air and drift travel through the cooling tower 2110.

The fan cowl extension 2144 has one or more side walls 2150 and the outer structure 2134 includes an upper wall 2154 and a side wall 2155. The side wall 2150, upper wall 2154 and side wall 2155 have interior surfaces that may have a high UV reflectivity due to the material of the side wall 2150, upper wall 2154, and side wall 2155. In one embodiment, the side wall 2150, upper wall 2154, and side wall 2155 may be made of the same or different and somewhat reflective materials such as galvanized steel, stainless steel, and aluminum.

During operation of the cooling tower 2110, drift, which are small water droplets, can be emitted from the outlet of fill sheets 2114 within the plenum 2136 and can make its way to the fan cowl extension 2144. As the drift evaporates, the drift may deposit solids, such as mineral deposits 2160, 2162, on the interior surfaces of the side wall 2150, upper wall 2154, and side wall 2155. The mineral deposits may decrease the UV reflectivity of the interior surfaces of the side wall 2150, upper wall 2154, and side walls 2155. The drift can also land on and produce mineral deposits on the UV light sources 2140 and 2142.

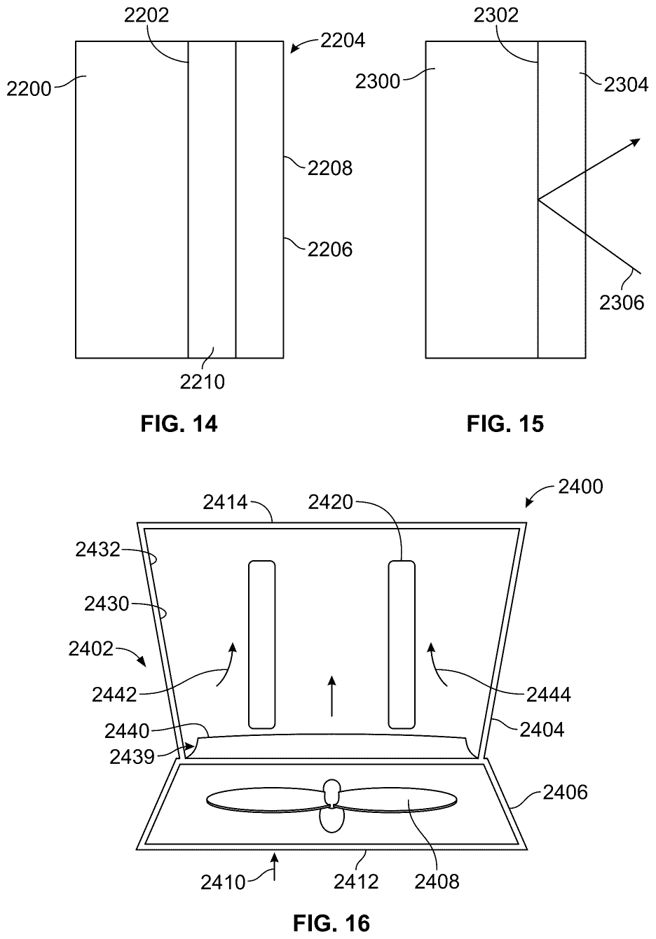

Regarding FIG. 14, a member 2200 of the cooling tower 2110 is shown in cross-section and has a surface 2202 with a given UV reflectivity. The member 2200 may be, for example, a wall, a strut, a baffle, a fan housing, or a liquid distribution system component. A UV reflector 2204 is attached to the member 2200 and includes a reflective layer 2206 with a reflective surface 2208 to provide UV reflectivity that is greater than the UV reflectivity of the member 2200 itself. The reflective layer 2206 may be connected to the surface 2202 via adhesive 2210. In another embodiment, the reflective layer 2206 is secured to the member 2200 via one or more fasteners, snap fits, or other mechanical connections. The member 2200 may be made of a material with a low UV reflectivity such as reinforced plastic or wood. The reflective layer 2206 may be made of a material selected to provide the reflective surface 2208 with a higher UV reflectivity than the surface 2202. The material of the reflective layer 2206 may be, for example an aluminum foil, aluminum foil tape, or aluminum or silver paint. Alternatively or additionally, the material of the reflective layer 2206 may for example include aluminum oxide, anodized aluminum, or a polymer such as PTFE or expanded PTFE. The UV reflector 2204 may take various shapes, such as an adhesive tape, a sheet that is mounted to the member 2200, or a reflector positioned by a mount. In this manner, the reflector 2204 may be connected to the member 2200 proximate a UV light source of the heat transfer apparatus to improve the UV irradiance within the cooling tower 2110. The UV reflector 2204 may cover the entirety of member 2200 or it may cover only part of member 2200. When the UV reflector 2204 covers only part of member 2200, it may have the benefit of reducing the cost of the UV reflector 2204. In cases where the UV light source is directional, the UV reflector 2204 may cover parts of the member 2200 which are exposed to UV light intensities above a threshold. For example, the UV reflector 2204 might cover only parts of member 2200 with a UV light intensity of at least 30% of the average UV light intensity on the member 2200.

Regarding FIG. 15, a member 2300 of the cooling tower 2110 is shown having a UV reflective surface 2302 with a nonstick layer 2304 thereon. The nonstick layer 2304 may be, for example, a layer of polytetrafluoroethylene (PTFE), silicone, silica, or a superhydrophobic coating. The nonstick layer 2304 permits UV rays 2306 to travel through the nonstick layer 2304 and reflect off the surface 2302 which preserves the reflectivity of the surface 2302. The nonstick layer 2304 limits adhesion of mineral scale to the surface 2302. The mineral scale may slide down or otherwise fall off of the nonstick layer 2304 upon the mineral scale being deposited on the nonstick layer 2304 by drift.

Regarding FIG. 16, an outlet fan assembly 2400 is provided that may be used with the cooling tower 2110. The outlet fan assembly 2400 includes a fan cowl 2402 having an upper portion 2404 and a lower portion 2406. The outlet fan assembly 2400 includes a fan 2408 that draws air in direction 2410 through an inlet opening 2412 of the lower portion 2406 and directs air out of an outlet opening 2414 of the fan cowl upper portion 2404. The outlet fan assembly 2400 includes one or more UV light sources 2420 to treat air flowing through the outlet fan assembly 2400. The fan cowl upper portion 2404 includes one or more side walls 2430 having one or more inner surface portions 2432 extending about a center of the fan cowl upper portion 2404. The outlet fan assembly 2400 includes an air flow shield 2439, such as a venturi 2440, to direct air flow generally in directions 2442, 2444 toward a center of the fan cowl upper portion 2404 and away from the inner surface portions 2432 of the fan cowl upper portion 2404. Because the venturi 2440 directs airflow inwardly away from the inner surface portions 2432, drift is kept away from the inner surface portions 2432 which reduces mineral scale buildup thereon. In another embodiment, the air flow shield 2439 includes one or more baffles to direct air away from the inner surface portions 2432.

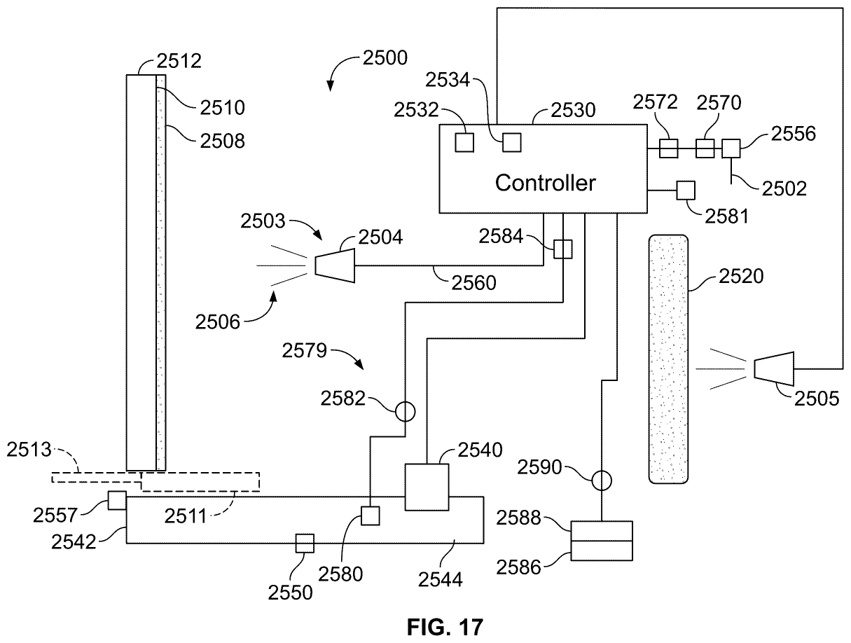

Regarding FIG. 17, an automated cleaning apparatus, such as cleaning apparatus 2500, is provided for a heat transfer apparatus such as the cooling tower 2100 or a residential HVAC system, as some examples. The cleaning apparatus 2500 is operable to remove dissolved solids that collect on an interior surface 2510 of the heat transfer apparatus and form the mineral deposits 2508. In one embodiment, the cleaning apparatus 2500 directs makeup water from a makeup water source 2502 to an outlet 2503 to remove solids such as dirt, debris, or mineral deposits 2508 from the surface 2510 of a member 2512 of a heat transfer apparatus. The member 2512 may be, for example, the side wall 2150, upper wall 2154, and/or side wall 2155 of the cooling tower 2110.

In this manner, by removing the mineral deposits 2508, a UV light from a UV light source 2520 may have a higher irradiation within the cooling tower which improves the effectiveness of the UV light source 2520 at cleaning the air flow within the cooling tower. Stated differently, the cleaning apparatus 2500 removes solids, such as mineral deposits and dirt, from the surface 2510 to provide a greater irradiance and the greater disinfecting power of the system for a given UV light source 2520.

In one embodiment, the outlet 2503 includes one or more nozzles 2504 configured to form a spray 2506 that removes solids from the surface 2510. In another embodiment, the outlet 2503 may include one or more sprinklers, drip manifolds, weir dams, or gravity flow distribution nozzles as some examples.

The cleaning apparatus 2500 may include a receptacle, such as a pan 2511, configured to collect the makeup water after the makeup water has been sprayed onto and removes solids from the surface 2510. The pan 2511 may direct the collected water into a sump 2542 and may include a filter to retain the solids in the pan 2511 for removal. By collecting the makeup water from the surface 2510, the pan 2511 keeps the makeup water from becoming drift within the heat exchange apparatus.

In another embodiment, the cleaning apparatus 2500 includes a drain 2513 that directs the makeup water and dissolved mineral deposits toward a disposal such as a sewer or storage sump associated with the cleaning apparatus 2500. The storage sump may be periodically emptied by service personnel. In this manner, the dissolved mineral scale and the chemical solution are kept out of the liquid 2544.

In one embodiment, the outlet 2503 includes one or more nozzles 2505 to form one or more sprays that removes solids, such as dirt and mineral deposits, from the UV light source 2520. For example, the nozzle 2505 sprays water on a member, such as lens, of the UV light source 2520. The controller 2530 may control one or more valves to selectively provide makeup water to the nozzle 2504 and/or nozzle 2505 based on whether the surface 2510 and/or the UV light source 2520 requires cleaning.

Alternatively or additionally, the UV light source 2520 is cleaned by water directed at the member 2512 but splashes or falls onto the UV light source 2520. In another embodiment, the UV light source 2520 is protected from direct contact by airflow within the heat transfer apparatus which inhibits drift from depositing solids on the UV light source 2520.

The cleaning apparatus 2500 has a controller 2530 with a processor 2532 and a memory 2534 that are operable to receive data from a biological activity sensor, such as a conductivity sensor 2540, associated with a sump 2542 of a heat transfer apparatus that includes the cleaning apparatus 2500. The processor 2532 may include, as examples, a microprocessor, an application-specific integrated circuit (ASIC), or a field programmable gate array (FPGA). The memory 2534 may include, as examples, an electrical charge-based storage media such as EEPROM or RAM, ROM, or other non-transitory computer readable media such as a flash memory device or magnetic or optical storage medium.

The sump 2542 collects a liquid 2544, such as evaporative fluid or a process fluid. The conductivity sensor 2540 detects conductivity of the liquid 2544. The conductivity sensor 2540 may be a 2-electrode, 4-electrode, or inductive sensor and may be analog or digital. The conductivity sensor 2540 may have a minimum conductivity measurement of about 0 to 500 microsiemens per centimeter (μS/cm). The conductivity sensor 2540 may have a maximum conductivity measurement of about 2,500 to 5,000 μS/cm. In response to a reduced airflow UV treatment effectiveness condition, such as the conductivity of the liquid 2544 exceeding a threshold, the controller 2530 opens a blowdown valve 2550 to drain the liquid 2544 from the sump 2542 and opens a flow control component such as a makeup valve 2556. There may be one or more conduits 2560 that direct the water from the makeup valve 2556 to the nozzle 2504. The controller 2530 keeps the makeup valve 2556 open, which causes the nozzle 2504 to discharge the spray 2506, while the blowdown valve 2550 drains the sump 2542 or after the sump 2542 has been drained. The makeup water from the nozzle 2504 both removes the mineral deposits 2508 from the surface 2510 and refills the sump 2542.

The reduced airflow UV treatment effectiveness condition utilized by the controller 2530 may include one or more conditions that indirectly affect the treatment provided by the UV light source 2520. For example, the controller 2530 may open the makeup valve 2556 in response to a level of water in the sump 2542 falling below a threshold as detected by a liquid level sensor 2557. The level of water in the sump 2542 may fall below the threshold such as due to evaporation of the liquid.

The cleaning apparatus 2500 may have components that improve the efficiency of the mineral deposit 2508 removing operation of the cleaning apparatus 2500. For example, in one embodiment the cleaning apparatus includes a heater 2572 to heat the water from the makeup valve 2556. The heater 2572 provides heated makeup water that will more readily absorb and melt mineral deposits 2508 from reflective surface 2510. Alternatively or additionally, the cleaning apparatus 2500 may include a liquid circulation subsystem 2579 including an inlet 2580 and a flow control component such as a pump 2582 that are operable to direct liquid in the sump 2542 to the nozzle 2504. The liquid circulation subsystem 2579 may include a heater 2584 for heating liquid from the sump 2542 before the liquid reaches the nozzle 2504.

In one embodiment, the cleaning apparatus 2500 utilizes a cleaning fluid 2586 in a reservoir 2588 to clean the surface 2510 instead of, or in addition to, water from the makeup water source 2502. For example, the cleaning fluid 2586 may contain a cleaning agent. A flow control component such as a pump 2590 is operable to direct the cleaning agent from the reservoir 2588 to the nozzle 2504 so that the cleaning agent may be sprayed onto the surface 2510 in addition to, or instead of, the makeup water from the makeup water source 2502 and/or the sump 2542.

In one embodiment, the cleaning apparatus 2500 utilizes a chemical solution in the reservoir 2588 to dissolve the mineral deposits 2508. The pan 2511 collects the chemical solution and a drain 2513 directs the used chemical solution toward a disposal such as a sewer or storage sump associated with the cleaning apparatus 2500. In this manner, the dissolved mineral scale and the chemical solution are kept out of the liquid 2544.

In one embodiment, the cleaning apparatus 2500 includes a flow control component such as a pump 2570 to increase the water pressure provided to the nozzle 2504 if the makeup water source 2502 has low water pressure.

The cleaning apparatus 2500 may direct the spray 2506 at the surface 2510 to remove the mineral deposits 2508 when the conductivity of the liquid 2544 is above a threshold, when the blowdown valve 2550 is opened to empty the sump 2542, and/or when the level of liquid 2544 in the sump 2542 is below a lower limit, which are all examples of reduced airflow UV treatment effectiveness conditions of the associated heat transfer apparatus. Alternatively or additionally, the controller 2530 may periodically direct the spray 2506 at the surface 2510 such as according to a timer or set schedule, which is another example of a reduced airflow UV treatment effectiveness condition. Still further, the controller 2530 may direct the spray 2506 at the surface 2510 in response to an event, such as a restart of the heat transfer apparatus and/or a user input from a remote computer requesting the controller 2530 perform a surface-cleaning operation, which are still further examples of reduced airflow UV treatment effectiveness conditions.

In one embodiment, the cleaning apparatus 2500 includes a UV sensor 2581 that detects the intensity of UV light within the heat transfer apparatus. The controller 2530 may utilize the detected UV light intensity to determine whether the surface 2510 and/or UV light source 2520 should be cleaned.

For example, the UV sensor 2581 may be positioned proximate the UV light source 2520 and the intensity of the UV light emitted by the UV light source 2520 decreases as dirt, mineral scale, and other substances accumulate on the UV light source 2520. The UV sensor 2581 may detect a radiant exposure or flux density of the UV light as indicative of the intensity of UV light. The controller 2530 may direct makeup water to the nozzle 2505 to clean the UV light source 2520 in response to the intensity of UV light detected by the UV sensor 2581 being at or below a lower threshold.

As another example, the UV sensor 2581 may be positioned near the surface 2510 to detect UV light reflected from the surface 2510. The controller 2530 may direct makeup water to the nozzle 2504 to clean the surface 2510 in response to the intensity of the reflected UV light being at or below a lower threshold. As another example, the UV sensor 2581 may be positioned farther away from the surface 2510 and UV light source 2520 to detect general UV irradiance within the heat transfer apparatus. The controller 2530 may direct makeup water to both nozzles 2504, 2505 to clean both the surface 2510 and the UV light source 2520 in response to the general UV irradiance detected by the UV sensor 2581 being at or below a lower threshold.

The controller 2530 may compare the intensity of UV light detected by the UV sensor 2581 before and after cleaning the surface 2510 and/or UV light source 2520. If the detected UV intensity is below a predetermined value after the cleaning has been performed, the controller 2530 may initiate an alarm indicative of the cleaning being ineffective, such as flashing a light of the heat transfer apparatus, communicating a notification to a remote computer, or causing the sending of an SMS text as a few examples.

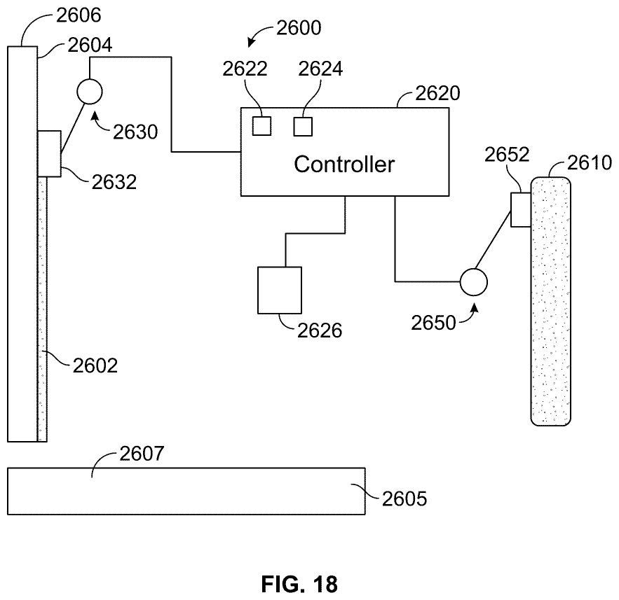

Regarding FIG. 18, another automated cleaning apparatus, such as cleaning apparatus 2600, is provided for removing solids, such as mineral deposits 2602, from a surface 2604 of a member 2606 of a heat transfer apparatus. By removing the mineral deposits 2602, the surface 2604 has a higher reflectivity which increases the UV irradiance within the heat transfer apparatus for a given UV light source 2610 and the associated effectiveness of the UV light source 2610.

The cleaning apparatus 2600 includes a controller 2620 having a memory 2622 and a processor 2624. The controller 2620 receives data from a UV sensor 2626. The UV sensor 2626 may measure the intensity of UV light within the heat transfer apparatus. The intensity of UV light within the heat rejection apparatus includes the UV light from a UV light source 2610 as well as UV light reflected by the surface 2604. In response to the UV intensity being at or below a lower threshold, the controller 2620 operates an actuator 2630 and causes a cleaning member 2632 to remove the solids, such as debris or mineral deposit 2602, from the surface 2604. The cleaning member 2632 may include, for example, a cloth, sponge, squeegee, brush, and/or wiper blade as some examples.

In one embodiment, the cleaning member 2632 includes a wiper blade and the actuator 2630 includes a motor to pivot the wiper blade. In another embodiment, the cleaning member 2632 includes a sponge and the actuator 2630 includes a linear actuator to shift the sponge back-and-forth. A maintenance worker may periodically clean and replace the cleaning member 2632.

The controller 2620 may periodically cause the cleaning member 2632 to clean the surface 2604 according to a schedule or a timer, which are examples of reduced airflow UV treatment effectiveness conditions. Alternatively or additionally, the controller 2620 may cause the cleaning member 2632 to clean the surface 2604 in response to an event such as startup of the heat transfer apparatus or the controller 2620 receiving a user command from a remote computer, which are further examples of reduced airflow UV treatment effectiveness conditions

In one embodiment, the cleaning apparatus 2600 includes an actuator 2650 operable to clean the UV light source with a cleaning member 2652. The actuator 2650 and cleaning member 2652 may be the same or different than the actuator 2630 and cleaning member 2632.

The controller 2620 operates the actuator 2650 to move the cleaning member 2652 along the UV light source 2610 and remove solids from the UV light source 2610. The controller 2620 may clean the UV light source 2610 periodically, such as after a predetermined operating time of the UV light source 2610.

Either or both of the cleaning apparatuses 2500, 2600 may be provided in a heat transfer apparatus such as cooling tower 2110. The heat transfer apparatus may have a plurality of a given type of cleaning apparatus 2500, 2600. For example, a heat transfer apparatus may have two or more cleaning apparatuses 2600 to clean two or more walls of the heat transfer apparatus and two or more cleaning apparatuses 2500 to clean two or more UV light sources of the heat transfer apparatus.

In another approach, either (or both) of the cleaning apparatuses 2500, 2600 may be provided as a retrofit kit for installation in an existing heat transfer apparatus. The retrofit kit including the cleaning apparatus 2500, 2600 may also include one or more UV light sources. As an example, a retrofit kit may be provided that includes the cleaning apparatus 2500 and the outlet fan assembly 2400 with UV light sources 2420 for being retrofit onto an existing heat transfer apparatus. The retrofit kit may include a spray nozzle, clamp assembly, water supply valve, and water supply piping which would allow installation of the spray nozzle to spray water and clean the UV light source and reflective surfaces on existing heat transfer apparatuses.

In one embodiment, the cleaning apparatuses 2500, 2600 may utilize the passage of time as a reduced airflow UV treatment effectiveness condition, such as the operating or run time of the associated heat transfer apparatus, to determine the UV light source(s) of the heat transfer apparatus requires cleaning. For example, the controllers 2530, 2620 may have a clock that is used to measure the run time of the heat transfer apparatus. When the run time exceeds a threshold, such as 100 hours, the controller 2530, 2620 initiates cleaning of the UV light source(s) of the heat transfer apparatus.

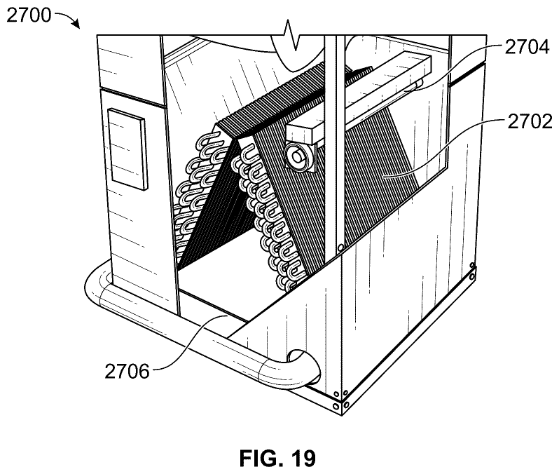

Regarding FIG. 19, a portion of a residential HVAC system 2700 is provided that includes an evaporator coil 2702 for cooling airflow and a UV treatment system 2704 to disinfect the cooled airflow. The UV treatment system 2704 also directs UV light at the exterior surfaces of the evaporator coil 2702 to disinfect the evaporator coil 2702. The residential HVAC system 2700 has a drain pan 2706 to collect water that condenses on the coil 2702.

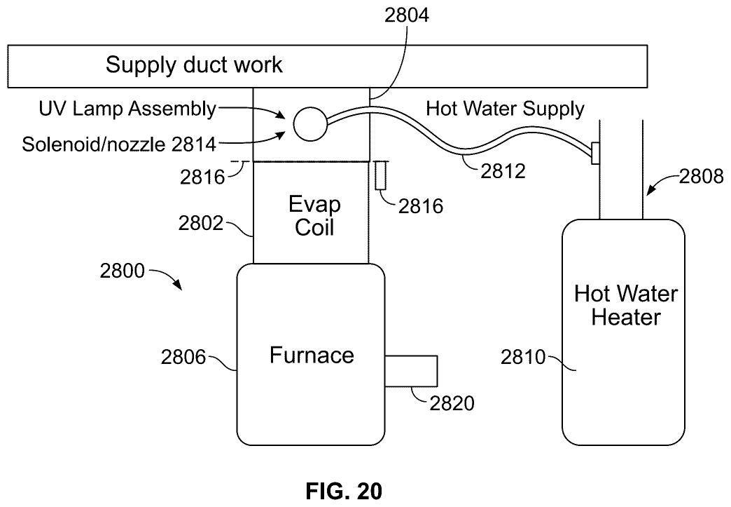

With reference to FIG. 20, a residential HVAC system 2800 is provided that is similar to the residential HVAC system 2700 and includes an evaporator coil 2802, a furnace portion 2804, a UV treatment system 2806, and a cleaning apparatus 2808. The cleaning apparatus 2808 utilizes a liquid, such as water, from a liquid source, such as a hot water heater 2810 or a water main, to clean the UV treatment system 2806. The residential HVAC system 2800 includes a receptacle such as a pan 2815 to collect the water or fluid that is used to clean the UV treatment system and a drain 2816 for directing the collected water or fluid toward a disposal such as a sewer of the residence.

In one embodiment, the cleaning apparatus 2808 has a conduit 2812 that provides hot water from the hot water heater 2810 to a flow control and distribution assembly 2814. The flow control and distribution assembly 2814 includes an outlet, such as a nozzle, configured to distribute hot water onto a UV light source, such as a UV lamp, of the UV treatment system 2806 to clean the UV light source by removing dust, dirt, and other debris from the UV light source. For example, the distributed hot water may wash accumulated debris off of lenses of UV lamps of the UV treatment system 2806. Alternatively or additionally, the flow control and distribution assembly 2814 may distribute, such as spray, hot water onto interior surfaces of sheet metal of the residential HVAC system 2800 proximate the UV treatment system 2806 to maintain a high level of reflectivity of the interior surfaces. The flow control and distribution assembly 2814 further includes a valve, such as a solenoid, to selectively direct hot water to the nozzle of the flow control and distribution assembly 2814.

The residential HVAC system 2800 includes, or is in communication with, a controller 2820 configured to operate the solenoid of the flow control and distribution assembly 2814 periodically, such as after a predetermined operating time of the UV treatment system 2806, for directing hot water to the nozzle of the flow control and distribution assembly 2814. The controller 2820 may direct the hot water to the UV source of the UV treatment system 2806 upon expiration of a period of time after the UV source has turned off, in order to give the UV source an opportunity to cool off before being cleaned.

Alternatively or additionally, the cleaning apparatus 2808 may include one or more UV sensors similar to the UV sensors 2581, 2626 discussed above to detect UV intensity within the residential HVAC system 2800. The controller 2820 may operate the solenoid to direct hot water to the nozzle of the flow control and distribution assembly 2814 and clean the UV light source in response to the detected UV intensity being at or below a lower threshold.

In one embodiment, the residential HVAC system 2800 may utilize a cleaning apparatus similar to the cleaning apparatus 2600 to clean internal surfaces of sheet metal of the residential HVAC system 2800 and/or the UV light source of the UV treatment system 2806.

Uses of singular terms such as “a,” “an,” are intended to cover both the singular and the plural, unless otherwise indicated herein or clearly contradicted by context. The terms “comprising,” “having,” “including,” and “containing” are to be construed as open-ended terms. It is intended that the phrase “at least one of” as used herein be interpreted in the disjunctive sense. For example, the phrase “at least one of A and B” is intended to encompass A, B, or both A and B.

While there have been illustrated and described particular embodiments of the present invention, it will be appreciated that numerous changes and modifications will occur to those skilled in the art, and it is intended for the present invention to cover all those changes and modifications which fall within the scope of the appended claims.

What is claimed is:

1. A heat transfer apparatus comprising:
an air inlet;
an air outlet;
a fan assembly to generate an airflow from the air inlet to the air outlet;
a heat exchanger to transfer heat to the airflow;
an ultraviolet (UV) light source operable to emit UV light and treat the airflow;
an automated cleaning apparatus operable to clean an interior surface of the heat transfer apparatus and increase an intensity of UV light in the heat transfer apparatus; and
a controller of the automated cleaning apparatus configured to cause the automated cleaning apparatus to clean the interior surface of the heat transfer apparatus in response to a reduced airflow UV treatment effectiveness condition.

2. The heat transfer apparatus of claim 1 wherein the UV light source includes the interior surface; and
wherein the controller is configured to cause the automated cleaning apparatus to clean the UV light source in response to the reduced airflow UV treatment effectiveness condition.

3. The heat transfer apparatus of claim 1 further comprising a light intensity sensor operable to detect a UV light intensity in the heat transfer apparatus; and
wherein the controller is configured to determine the reduced airflow UV treatment effectiveness condition based at least in part upon the UV light intensity.

4. The heat transfer apparatus of claim 1 further comprising a light intensity sensor operable to detect an intensity of the UV light source, the controller configured to:
compare the intensity of the UV light source to a threshold UV intensity; and
determine the reduced airflow UV treatment effectiveness condition based at least in part upon the intensity of the UV light source being less than the threshold UV intensity.

5. The heat transfer apparatus of claim 1 further comprising a biological activity sensor operatively connected to the controller, the controller configured to determine the reduced airflow UV treatment effectiveness condition based at least in part upon data from the biological activity sensor.

6. The heat transfer apparatus of claim 1 wherein the controller is configured to monitor an operating time of the heat transfer apparatus; and
wherein the controller is configured to determine the reduced airflow UV treatment effectiveness condition based at least in part upon the operating time of the heat transfer apparatus exceeding a predetermined time period.

7. The heat transfer apparatus of claim 1 wherein the automated cleaning apparatus includes an inlet to receive a liquid from a liquid source and an outlet to provide the liquid to the interior surface, the controller configured to cause the outlet to provide the liquid to the interior surface to clean the interior surface in response to the reduced airflow UV treatment effectiveness condition.

8. The heat transfer apparatus of claim 7 further comprising a liquid makeup line to provide the liquid to the heat transfer apparatus; and
wherein the inlet of the automated cleaning apparatus is connected to the liquid makeup line; and
wherein the automated cleaning apparatus includes a valve and/or a pump to direct the liquid to the outlet of the automated cleaning apparatus.

9. The heat transfer apparatus of claim 7 wherein the heat exchanger includes a plurality of heat exchange elements, a liquid distribution system operable to distribute the liquid onto the heat exchange elements, and a receptacle to collect the liquid from the heat exchange elements; and
wherein the inlet of the automated cleaning apparatus is connected to the receptacle to receive the liquid from the receptacle.

10. The heat transfer apparatus of claim 9 wherein the heat exchange elements include at least one of serpentine coils, fill, and adiabatic pads.

11. The heat transfer apparatus of claim 1 wherein the automated cleaning apparatus includes a cleaning member and an actuator, the controller configured to cause the actuator to move the cleaning member relative to the interior surface and clean the interior surface in response to the reduced airflow UV treatment effectiveness condition.

12. The heat transfer apparatus of claim 11 wherein the cleaning member includes at least one of:
a cloth;
a sponge;
a squeegee;
a brush; and
a wiper blade.

13. The heat transfer apparatus of claim 1 wherein the heat exchanger includes heat exchanger elements, a first liquid distribution system operable to distribute a first liquid onto the heat exchanger elements, and a first receptacle to collect the first liquid;
wherein the automated cleaning apparatus includes an outlet to provide a second liquid to the interior surface, the controller configured to cause the outlet to provide the second liquid to the interior surface to clean the interior surface in response to the reduced airflow UV treatment effectiveness condition; and
a second receptacle to collect the second liquid after the second liquid has been provided to the interior surface, the second receptacle inhibiting the collected second liquid from entering the first receptacle.

14. The heat transfer apparatus of claim 1 wherein the automated cleaning apparatus is configured to receive a liquid from a liquid source;
wherein the automated cleaning apparatus includes a heater to heat the liquid; and
wherein the controller is configured to cause the heater to heat the fluid and cause the automated cleaning apparatus to provide the heated liquid to the interior surface to clean the interior surface.

15. The heat transfer apparatus of claim 1 further comprising an outer structure, wherein the heat exchanger and the UV light source are in the outer structure; and
wherein the interior surface comprises an interior surface of a wall of the outer structure.

16. The heat transfer apparatus of claim 1 further comprising a cowl; and
wherein the interior surface comprises an interior surface of the cowl.

17. The heat transfer apparatus of claim 1 further comprising a process fluid inlet to receive a process fluid and a process fluid outlet to return the process fluid; and
wherein the heat exchanger is configured to transfer heat from the process fluid to the airflow.

18. The heat transfer apparatus of claim 1 wherein the reduced airflow UV treatment effectiveness condition includes a reduced airflow UV effectiveness condition of the UV light source.
